(19) United States
(12) Patent Application Publication
(10) Pub. No.: US 2016/0220277 A1
(43) Pub. Date: Aug. 4, 2016

(54) SPINAL IMPLANT SYSTEM AND METHODS OF USE

(71) Applicant: Warsaw Orthopedic, Inc., Warsaw, IN (US)

(72) Inventors: William Alan Rezach, Atoka, TN (US); Jason May, Cordova, TN (US); Rodney Ballard, Lakeland, TN (US)

(21) Appl. No.: 14/614,073

(22) Filed: Feb. 4, 2015

(51) Int. Cl. A61B 17/70

(52) U.S. Cl. CPC A61B 17/7032

(57) ABSTRACT

A spinal implant system comprises a plurality of alternate first members. Each of the first members includes an inner surface defining an implant cavity. A second member is configured to penetrate tissue and includes a mating element engageable with a first member such that the second member is interchangeable with the plurality of first members. The second member is manually engageable with the first member to connect the members. Fasteners, instruments and methods are disclosed.

### CROSS-REFERENCE TO RELATED APPLICATION

This application claims the benefits of U.S. Provisional Patent Application No. Ser. No. 61/951,382 (Attorney Docket No. C00007248.USP1) filed Mar. 11, 2014 and U.S. Provisional Patent Application No. Ser. No. 61/951,401 (Attorney Docket No. C00007252.USP1) filed Mar. 11, 2014, the contents of each of these references being hereby incorporated in their entireties by reference.

### TECHNICAL FIELD

The present disclosure generally relates to medical devices for the treatment of spinal disorders, and more particularly to a surgical implant system including a bone fastener.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. As part of these surgical treatments, spinal constructs such as vertebral rods are often used to provide stability to a treated region. Rods redirect stresses away from a damaged or defective region while healing takes place to restore proper alignment and generally support the vertebral members. During surgical treatment, one or more rods and bone fasteners can be delivered to a surgical site. The rods may be attached via the fasteners to the exterior of two or more vertebral members. This disclosure describes an improvement over these prior art technologies.

### SUMMARY

In one embodiment, a spinal implant system is provided. The spinal implant system comprises a plurality of alternate first members. Each of the first members includes an inner surface defining an implant cavity. A second member is configured to penetrate tissue and includes a mating element engageable with a first member such that the second member is interchangeable with the plurality of first members. The second member is manually engageable with the first member to connect the members. In some embodiments, fasteners, instruments and methods are disclosed.

### BRIEF DESCRIPTION OF THE DRAWINGS

The present disclosure will become more readily apparent from the specific description accompanied by the following drawings, in which:

FIG. 1 is a perspective view of components of one embodiment of a spinal implant system in accordance with the principles of the present disclosure;

FIG. 2 is a cross section view of the components shown in FIG. 1;

FIG. 3 is a cross section view of the components shown in FIG. 1;

FIG. 4 is a break away view of components of the system shown in FIG. 1;

FIGS. 5A-5E are cross section views of components of one embodiment of a spinal implant system illustrating assembly;

FIG. 6 is a cross section view of components of one embodiment of a spinal implant system in accordance with the principles of the present disclosure; and

FIG. 7 is a cross section view of components of one embodiment of a spinal implant system in accordance with the principles of the present disclosure.

### DETAILED DESCRIPTION

The exemplary embodiments of a surgical system and related methods of use disclosed are discussed in terms of medical devices for the treatment of musculoskeletal disorders and more particularly, in terms of a spinal implant system including a bone fastener. In one embodiment, the spinal implant system includes an implant comprising a bone fastener, such as, for example, a universal pedicle bone screw. In some embodiments, the spinal implant system includes a selectively coupled pedicle screw system that allows for operating room back-table assembly of a bone fastener without use of instrumentation.

In some embodiments, the spinal implant system comprises a modular system includes a bone fastener including an array of members, such as, for example, receivers that can be selectively coupled to members, such as, for example, bone screw shafts. In some embodiments, the spinal implant system comprises a selectively coupled bone fastener that can be assembled on a surgical table or in-situ. In some embodiments, the selectively coupled bone fastener is assembled with a force of less than 50 Newtons (N). In some embodiments, the bone fastener is selectively coupled with a non-instrumented assembly. In some embodiments, the non-instrumented assembly comprises manually engaging a screw shaft with a head/receiver of the bone fastener. In some embodiments, the non-instrumented assembly comprises manually engaging the screw shaft in a pop-on engagement with the head/receiver of the bone fastener. In some embodiments, a force required to manually engage a screw shaft with a head/receiver of the bone fastener in a non-instrumented assembly is in a range of 2 to 50 N. In some embodiments, a force required to manually engage a screw shaft with a head/receiver of the bone fastener in a non-instrumented assembly is in a range of 5 to 10 N. In some embodiments, a screw shaft is manually engaged with a head/receiver of the bone fastener in a non-instrumented assembly, as described herein, such that removal of the head/receiver from the screw shaft requires a force and/or a pull-out strength of at least 5000 N. In some embodiments, this configuration provides manually engageable components of a bone fastener that are assembled without instrumentation, and subsequent to assembly, the assembled components have a selected pull-out strength and/or can be pulled apart, removed and/or separated with a minimum required force.

In some embodiments, the bone fastener includes a ring disposed with a receiver connected with a screw shaft. In some embodiments, the ring is configured to snap onto the screw shaft. In some embodiments, the ring has a minimized thickness and/or height to facilitate snapping the ring onto the screw shaft. In some embodiments, the force required to snap the ring onto the screw shaft is in a range of 2 to 50 N. In some embodiments, the force required to snap the ring onto the screw shaft is in a range of 5 to 10 N.

In some embodiments, the bone fastener is configured for assembly without the use of an instrument, such as, for example, a practitioner, surgeon and/or medical staff utilizes their hands for assembly. In some embodiments, the system requires minimal force to attach a receiver and a shaft in-situ thereby reducing a pre-load on the vertebrae, such as, for, example, the pedicle. In some embodiments, the bone fastener includes a receiver having a double ring chamber. In some embodiments, the bone fastener includes an expandable ring.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed spinal implant system may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The present disclosure may also be alternatively employed with procedures for treating the lumbar, cervical, thoracic, sacral and pelvic regions of a spinal column. The spinal implant system of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

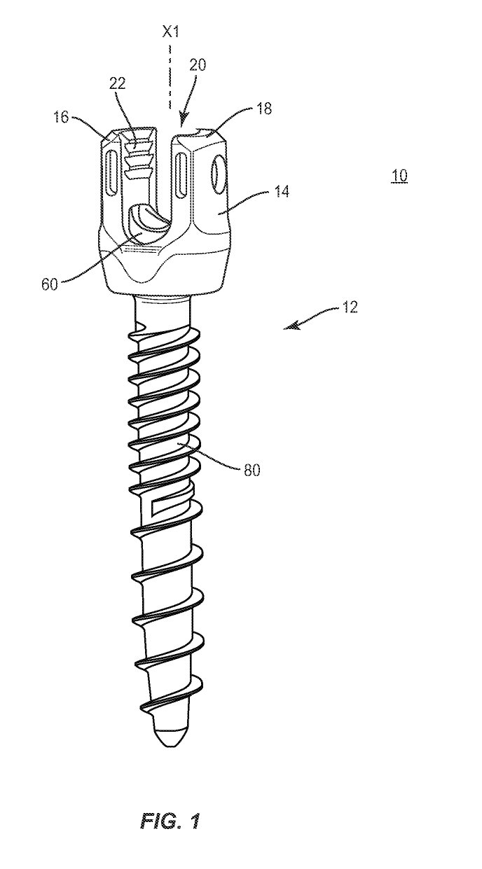

The following discussion includes a description of a surgical system including a bone fastener, related components and methods of employing the surgical system in accordance with the principles of the present disclosure. Alternate embodiments are also disclosed. Reference is made in detail to the exemplary embodiments of the present disclosure, which are illustrated in the accompanying figures. Turning to FIGS. 1-4, there are illustrated components of a spinal implant system 10 including a plurality of alternate bone fastener configurations, such as, for example, a plurality of bone screw configurations 12.

The components of spinal implant system 10 can be fabricated from biologically acceptable materials suitable for medical applications, including metals, synthetic polymers, ceramics and bone material and/or their composites. For example, the components of spinal implant system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TOP), hydroxyapatite (HA)-TCP, calcium sulfate, or other resorbable polymers such as polyaetide, polyglycolide, polytyrosine carbonate, polycaroplaetohe and their combinations.

Various components of spinal implant system 10 may have material composites, including the above materials, to achieve various desired characteristics such as strength, rigidity, elasticity, compliance, biomechanical performance, durability and radiolucency or imaging preference. The components of spinal implant system 10, individually or collectively, may also be fabricated from a heterogeneous material such as a combination of two or more of the above-described materials. The components of spinal implant system 10 may be monolithically formed, integrally connected or include fastening elements and/or instruments, as described herein.

In some embodiments, spinal implant system 10 comprises a spinal implant kit, which includes a plurality of members, such as, for example, implant receivers 14. Receiver 14 is configured for selection from the plurality of receivers such that receiver 14 is connectable with an interchangeable member, such as, for example, a shaft 80. In some embodiments, receiver 14 is configured for selection from the plurality of receivers such that receiver 14 is connectable with a compatible shaft 80.

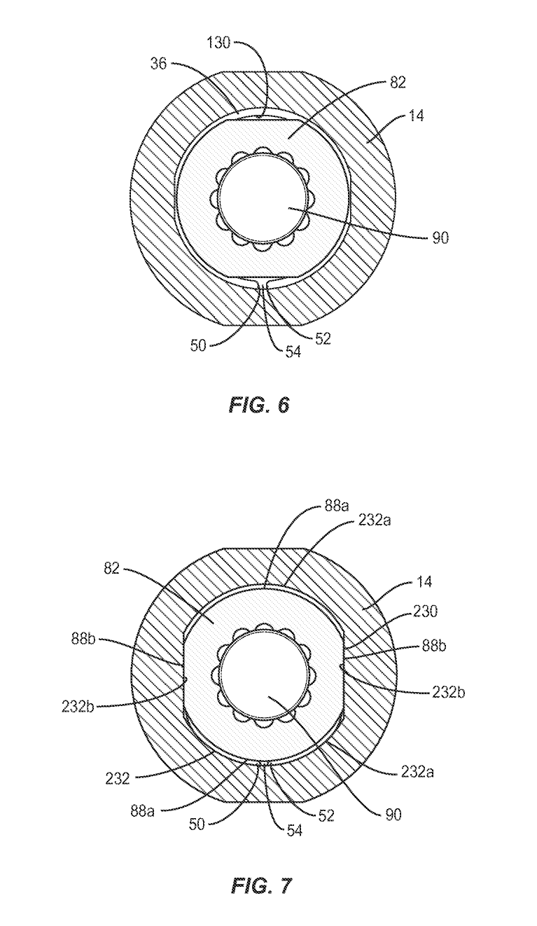

An interchangeable mating element, such as, for example, a head 82 of shaft 80 is interchangeable with a mating dement, as described herein, of each of the plurality of receivers 14 to form a selected bone screw 12 having a selected movement of its components parts and/or movement relative to tissue. In some embodiments, the selected movement includes rotation and/or pivotal movement of shaft 80 relative to receiver 14 about one or a plurality of axes. In some embodiments, the selected movement includes rotation and/or pivotal movement of shaft 80 relative to receiver 14 through one or a plurality of planes. In some embodiments, shaft 80 is connected to a selected receiver 14 to comprise a multi-axial fastener, as shown in FIG. 6. In some embodiments, shaft 80 is connected to a selected receiver 14 to comprise a uni-axial fastener, as shown in FIG. 7. In some embodiments, spinal implant system 10 comprises a spinal implant kit, which includes receivers 14 and alternate receivers, such as those described herein.

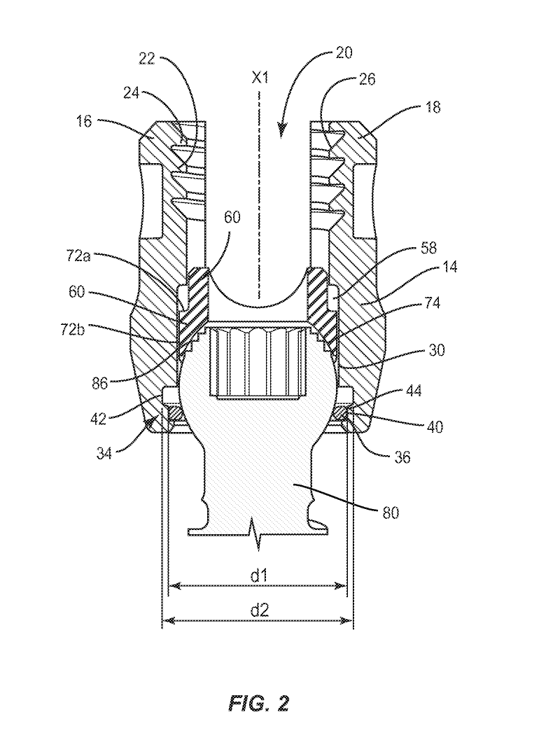

Each receiver 14 extends along and defines an axis X1, as shown in FIGS. 1 and 2. Each receiver 14 includes a pair of spaced apart arms 16, 18 that define an implant cavity 20 therebetween configured for disposal of a component of a spinal construct, such as, for example, a spinal rod (not shown). Arms 16, 18 each extend parallel to axis X1. In some embodiments, arm 16 and/or arm 18 may be disposed at alternate orientations, relative to axis X1, such as, for example, transverse, perpendicular and/or other angular orientations such as acute or obtuse, coaxial and/or may be offset or staggered. Arms 16, 18 each include an arcuate outer surface extending between a pair of side surfaces. At least one of the outer surfaces and the side surfaces of arms 16, 18 have at least one recess or cavity therein configured to receive an insertion tool, compression instrument and/or instruments for inserting and tensioning bone screw 12. In some embodiments, arms 16, 18 are connected at proximal and distal ends thereof such that receiver 14 defines a closed spinal rod slot.

Cavity 20 is substantially U-shaped. In some embodiments, all or only a portion of cavity 20 may have alternate cross section configurations, such as, for example, closed, V-shaped, VV-shaped, oval, oblong triangular, square, polygonal, irregular, uniform, non-uniform, offset, staggered, and/or tapered. Each receiver 14 includes an inner surface 22. A portion of surface 22 includes a thread form 24 located adjacent arm 16 and a thread form 26 located adjacent arm 18. Thread forms 24, 26 are each configured for engagement with a coupling member, such as, for example, a setscrew (not shown), to retain a spinal construct, such as, for example, a spinal rod (not shown) within cavity 20. In some embodiments, surface 22 may be disposed with the coupling member in alternate fixation configurations, such as, for example, friction fit, pressure fit, locking protrusion/recess, locking keyway and/or adhesive. In some embodiments, all or only a portion of surface 22 may have alternate surface configurations to enhance engagement with the spinal rod and/or the setscrew such as, for example, rough, arcuate, undulating, mesh, porous, semi-porous, dimpled and/or textured. In some embodiments, receiver 14 may include alternate configurations, such as, for example, closed, open and/or side access. Other alternate configurations of the receiver 14 may allow, for example, an offset that may retain a spinal construct away from the head 82. The offset may extend medially, laterally, or in any other orientation relative to the head 82. The spinal construct may be received within the cavity 20 in any orientation, for example, in a side-loaded configuration, a top-loaded configuration or an oblique configuration.

A portion of surface 22 of each receiver 14 defines a particularly configured mating element, such as, for example, an engagement surface 30 configured to interface in a selective mating engagement with head 82 of shaft 80. In some embodiments, engagement surface 30 includes flats and/or arcuate surfaces to form various bone screw configurations, such as, for example, multi-axial screws, sagittal angulation screws, pedicle screws, mono-axial screws, uni-planar screws, fixed screws, anchors, tissue penetrating screws, conventional screws, expanding screws. In one embodiment, as shown in FIG. 6, head 82 is slidably engageable with a surface 130, similar to surface 30 described herein, and movable relative thereto such that shaft 80 is rotatable along a plurality of axes relative to receiver 14 including rotation about axis X1. As such, interchangeable shaft 80 is connected with a selected receiver 14 from the kit of receivers 14 to form a multi-axial bone screw 12. In one embodiment, as shown in FIG. 7, head 82 is slidably engageable with a surface 230, similar to surface 30 described herein, which includes a keyway 232 that includes mating elements, such as, for example, arcuate surfaces 232a and planar surfaces, such as, for example, flats 232b. Flats 232b are configured to interface with flats 88b of head 82 and arcuate surfaces 232a are configured to interface with arcuate surfaces 88a in a keyed connection such that shaft 80 is rotatable along a single axis and/or within a single plane relative to receiver 14. Flats 232b engage flats 88b to resist and/or prevent rotation of receiver 214 about a selected axis.

Each surface 22 defines a cavity, such as, for example, a groove 34 configured for disposal of a band, such as, for example, a circumferential ring 36, as shown in FIGS. 2-4. Groove 34 includes a portion, such as for, example, a circumferential channel 40 having a diameter d1 and a portion, such as for, example, a circumferential channel 42 having a diameter d2 that is greater than diameter d1. Channel 42 is adjacent and proximal to channel 40. Channel 42 is separated from channel 40 by a protrusion, such as, for example, a lip 44. In some embodiments, shaft 80 is manually engageable with receiver 14 and/or shaft 80 is coupled with receiver 14 in a non-instrumented assembly such that ring 36 translates from and into channels 40, 42, and over lip 44, as described herein.

In some embodiments, manual engagement and/or non-instrumented assembly of receiver 14 and shaft 80 includes the coupling of receiver 14 and shaft 80 without use of separate and/or independent instrumentation engaged with bone fastener 12 components to effect assembly. In some embodiments, manual engagement and/or non-instrumented assembly includes a practitioner, surgeon and/or medical staff grasping receiver 14 and shaft 80 and forcibly assembling the components of bone fastener 12. In some embodiments, manual engagement and/or non-instrumented assembly includes a practitioner, surgeon and/or medical staff grasping receiver 14 and shaft 80 and forcibly snap fitting the components of bone fastener 12 together, as described herein. In some embodiments, manual engagement and/or non-instrumented assembly includes a practitioner, surgeon and/or medical staff grasping receiver 14 and shaft 80 and forcibly pop fitting the components of bone fastener 12 together and/or pop fitting receiver 14 onto shaft 80, as described herein. In some embodiments, a force in a range of 2-50 N is required to manually engage receiver 14 and shaft 80 and forcibly assemble the components of bone fastener 12. For example, a force in a range of 2-50 N is required to snap fit and/or pop fit assemble receiver 14 and shaft 80. In some embodiments, a force in a range of 5-10 N is required to manually engage receiver 14 and shaft 80 and forcibly assemble the components of bone fastener 12. For example, a force in a range of 5-10 N is required to snap fit and/or pop fit assemble receiver 14 and shaft 80. In some embodiments, shaft 80 is manually engaged with receiver 14 in a non-instrumented assembly, as described herein, such that removal of receiver 14 from shaft 80 requires a force and/or a pull-out strength of at least 5000 N. In some embodiments, this configuration provides manually engageable components of bone fastener 12 that are assembled without instrumentation, and subsequent to assembly, the assembled components have a selected pull-out strength and/or can be pulled apart, removed and/or separated with a minimum required force,

Ring 36 includes a height h and a width w. Ring 36 includes a circumference C that extends between end 50 and end 52. Ends 50, 52 define an opening, such as, for example, a gap 54. In some embodiments, gap 54 is sized such that gap 54 has a thickness t that is less than height h and width w. Ring 36 is expandable and resilient between a contracted and/or capture orientation, and an expanded orientation, as described herein. Ring 36 facilitates manual engagement of a selected receiver 14 and shaft 80 such that the selected receiver 14 is attached with shaft 80 in a non-instrumented assembly, as described herein.

In some embodiments, each surface 22 includes a cavity, such as, for example, a slot 58 configured to receive a flange of a crown 60, as discussed herein. Slot 58 includes surfaces 58a, 58b. In some embodiments, all or only a portion of each surface 22 may have alternate surface configurations to enhance engagement with the spinal rod and/or the setscrew such as, for example, rough, arcuate, undulating, mesh, porous, semi-porous, dimpled and/or textured.

Crown 60 is configured for disposal within cavity 20 of the selected receiver 14. Crown 60 includes a wall 62 having an end surface 64 and an end surface 66. Surface 64 is configured to define at least a portion 68 of cavity 20. Portion 68 is defined by an outer surface 70 that defines a curved portion of crown 60 configured for engagement with a spinal rod. In some embodiments, all or only a portion of surface 70 may have alternate cross section configurations, such as, for example, oval, oblong triangular, square, polygonal, irregular, uniform, non-uniform, offset, staggered, and/or tapered.

Surface 64 defines a receiver engagement portion, such as, for example, a flange 72 configured for mating engagement with slot 54 of the selected receiver. Flange 72 includes surfaces 72a, 72b. In some embodiments, flange 72 engages the surface of slot 54 in a keyed connection such that surface 72a abuts surface 54a and surface 72b abuts surface 54b. In some embodiments, engagement of flange 72 and slot 54 prevents rotation and/or axial translation of crown 60 relative to surface 22 of the selected receiver 14. Surface 70 is disposed in fixed alignment with surface 22 for disposal of a spinal rod. Surface 66 defines an engagement portion 74 configured for engagement with head 82, as described herein.

Shaft 80 is configured to penetrate tissue, such as, for example, bone. Head 82 is interchangeably engageable with any of the plurality of receivers 14. Head 82 includes a substantially spherical proximal portion configured for moveable disposal with the selected receiver 14 and crown 60. Head 82 includes a surface 84 that defines a plurality of ridges 86 to improve purchase of head 82 with crown 60. Head 82 includes a portion 87 having a maximum diameter d3. Portion 87 is configured to apply a force to ring 36 to move ring 36 between a contracted and/or capture orientation and an expanded orientation, as described herein. Engagement portion 74 of crown 60 is concave or semi-spherical to accommodate the substantially spherical configuration of head 82 such that head 82 is rotatable relative to receiver 14.

In some embodiments, head 82 is slidably engageable with surface 30 and movable relative thereto such that shaft 80 is rotatable along a plurality of axes relative to receiver 14 including rotation about axis X1. In some embodiments, surface 84 includes interchangeable mating surfaces, such as for example, arcuate portions and/or planar portions configured for disposal with surface 30 of any of the plurality of receivers 14 to limit rotation of receiver 14 relative to shaft 80. Interchangeable shaft 80 is connected with a selected receiver 14 from the kit of receivers 14 to form a bone screw 12. In some embodiments, receiver 14 may be disposed with shaft 80 in alternate fixation configurations, such as, for example, friction fit, pressure fit, locking protrusion/recess, locking keyway and/or adhesive.

Head 82 includes a socket 90 having a hexalobe geometry configured for disposal of a similarly shaped bit of a tool, such as, for example, a driver (not shown) to engage the driver with head 82 to rotate shaft 80. Socket 90 is in communication with cavity 20 such that a driver may be inserted between arms 16, 18 and translated axially, until the bit of the driver is disposed in socket 90. In some embodiments, socket 90 has a cruciform, phillips, square, hexagonal, polygonal, star cross sectional configuration configured for disposal of a correspondingly shaped portion of a driver.

In assembly, operation and use, spinal implant system 10, similar to the systems and methods described herein, includes a selected bone screw 12, which comprises a selected receiver 14 for connection with interchangeable shaft 80 having a selected movement, and is employed with a surgical procedure for treatment of a spinal disorder affecting a section of a spine of a patient, as discussed herein. Spinal implant system 10 is employed with a surgical procedure for treatment of a condition or injury of an affected section of the spine. In some embodiments, a selected bone screw 12 comprises a selected receiver 14 for connection with a compatible shaft 80.

The components of spinal implant system 10 include a spinal implant kit, which comprises the plurality of receivers and interchangeable shafts 80. In some embodiments, spinal implant system 10 includes a spinal implant kit, which comprises the plurality of receivers and compatible shafts 80. The plurality of receivers include receivers 14 and alternate receivers, such as those described herein, that interface with interchangeable shafts 80 to comprise one or more bone screw configurations. Selected bone screws 12 and one or a plurality of spinal implants, such as, for example, vertebral rods can be delivered or implanted as a pre-assembled device or can be assembled in situ. The components of spinal implant system 10 may be may be completely or partially revised, removed or replaced.

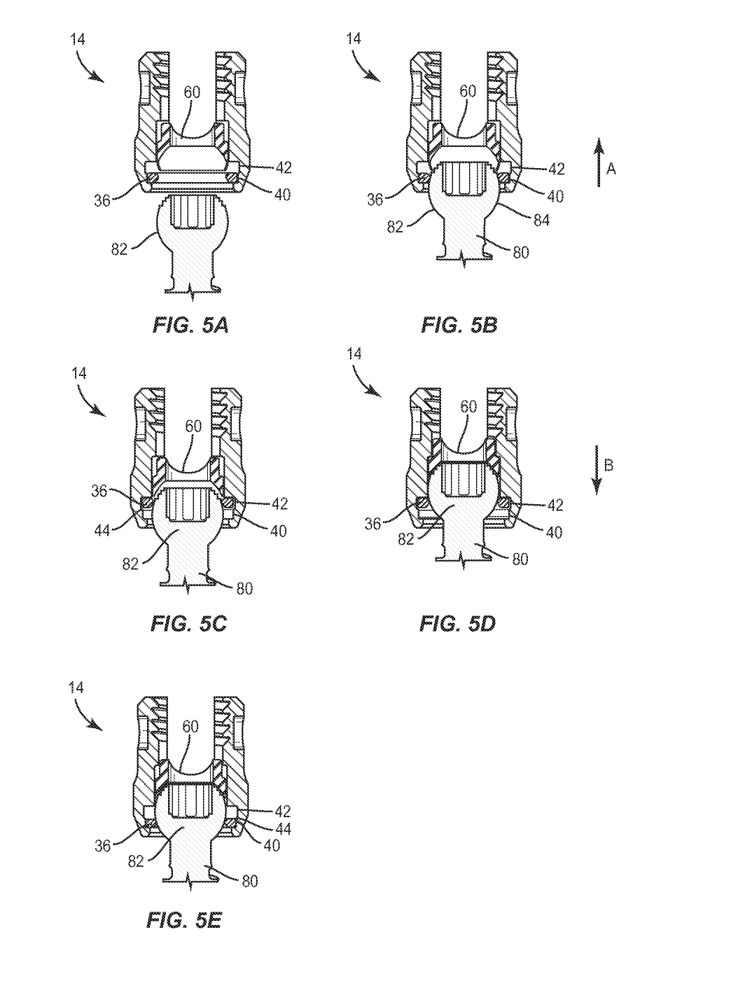

In some embodiments, a receiver 14 is selected from the kit of the plurality of receivers 14 for interchangeable connection with shaft 80 to comprise a bone screw 12 having a selected movement. In some embodiments, the kit of receivers 14 includes a variety of receivers having different movement configurations when assembled with an interchangeable shaft, such as, for example, multi-axial movement, sagittal angulation movement, fixed axis movement, mono-axial movement and/or uni-planar movement. In some embodiments, crown 60 is disposed with the selected receiver 14 such that flange 72 engages slot 58, as described herein. In some embodiments, ring 36 is disposed with the selected receiver 14 such that circumference C is disposed in channel 40 in a contracted and/or capture orientation having a diameter d1, as shown in FIGS. 2 and 5A.

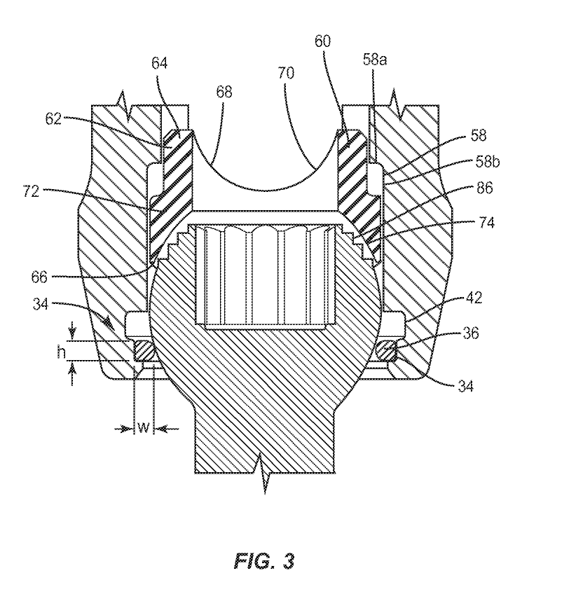

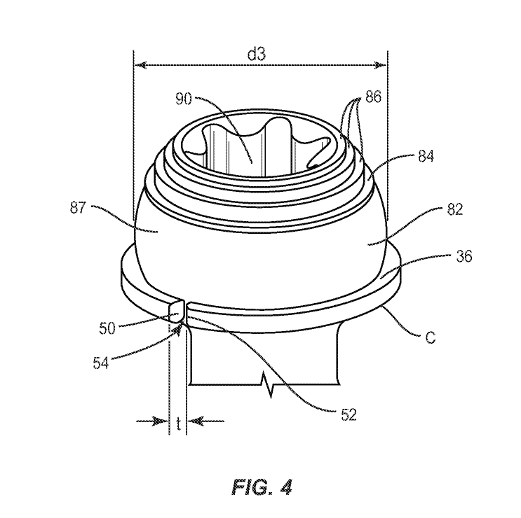

Shaft 80 is manually engageable, as described herein, with selected receiver 14, as shown in FIG. 5B, such that ring 36 translates in a first direction, as shown by arrow A, from channel 40 into channel 42 over lip 44, as shown in FIG. 50. As head 82 engages ring 36, ring 36 expands to an expanded orientation, as shown in FIG. 5D, such that head 82 passes through ring 36. In the expanded orientation, ring 36 expands to diameter d2 (FIG. 2) in channel 42. Shaft 80 continues to translate causing ridges 86 to engage surface 74. As shaft 80 translates into engagement with crown 60, the resiliency of ring 36 causes ring 36 to contract and translate along surface 84 of head 82, in the direction shown by arrow B in FIG. 5D, to a position distal to portion 87. As ring 36 contracts back to the capture orientation, ring 36 translates over lip 44 into channel 40, as shown in FIG. 5E. Diameter d3 (FIG. 4) of portion 86 prevents head 82 from moving through ring 36 when ring 36 returns to channel 40. In some embodiments, ring 36 is disposed with head 82 to enhance a retaining strength of bone screw 12 and resist and/or prevent shearing of shaft 80. In some embodiments, ring 36 is expandable with a force in a range of 2-50 N. In some embodiments, ring 36 is expandable with a force in a range of 5-10 N. In some embodiments, manual engagement of selected receiver 14 and shaft 80 includes snap fit and/or pop fit assembly of receiver 14 and shaft 80, as described herein. In some embodiments, shaft 80 is assembled with receiver 14, as described herein, such that removal of receiver 14 from shaft 80 requires a force and/or a pull-out strength of at least 5000 N.

In use, for treatment of a spinal disorder, shaft 80 can be threaded and engaged with tissue. In some embodiments, the selected bone screw 12 is disposed adjacent vertebrae at a surgical site and is manipulated to drive, torque, insert or otherwise connect bone screw 12 with vertebrae.

In some embodiments, spinal implant system 10 includes an agent, which may be disposed, packed, coated or layered within, on or about the components and/or surfaces of spinal implant system 10. In some embodiments, the agent may include bone growth promoting material, such as, for example, bone graft to enhance fixation of the fixation elements with vertebrae. In some embodiments, the agent may be hydroxyapatite coating. In some embodiments, the agent may include one or a plurality of therapeutic agents and/or pharmacological agents for release, including sustained release, to treat, for example, pain, inflammation and degeneration.

In some embodiments, the use of microsurgical and image guided technologies may be employed to access, view and repair spinal deterioration or damage, with the aid of spinal implant system 10. The components of spinal implant system 10 can be made of radiolucent materials such as polymers. Radiomarkers may be included for identification under x-ray, fluoroscopy, CT or other imaging techniques.

In some embodiments, spinal implant system 10 can include one or a plurality of bone screws 12 such as those described herein and/or fixation elements, which may be employed with a single vertebral level or a plurality of vertebral levels. In some embodiments, bone screws 12 may be engaged with vertebrae in various orientations, such as, for example, series, parallel, offset, staggered and/or alternate vertebral levels. In some embodiments, bone screws 12 may be configured as multi-axial screws, sagittal angulation screws, pedicle screws, mono-axial screws, uniplanar screws, fixed screws, anchors, tissue penetrating screws, conventional screws, expanding screws. In some embodiments, bone screws 12 may be employed with wedges, anchors, buttons, clips, snaps, friction fittings, compressive fittings, expanding rivets, staples, nails, adhesives, posts, connectors, fixation plates and/or posts.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal implant system comprising:
a plurality of alternate first members, each of the first members including an inner surface defining an implant cavity; and
a second member being configured to penetrate tissue and including a mating element engageable with a first member such that the second member is interchangeable with the plurality of first members,
the second member being manually engageable with the first member to connect the members.

2. A spinal implant system as recited in claim 1, wherein the members are engageable in a snap-fit assembly.

3. A spinal implant system as recited in claim 1, wherein the members are engageable in a pop-on assembly.

4. A spinal implant system as recited in claim 1, wherein a force in a range of 2 through 50 N is applied to the members to manually engage and connect the members.

5. A spinal implant system as recited in claim 1, wherein a force in a range of 5 through 10 N is applied to the members to manually engage and connect the members.

6. A spinal implant system as recited in claim 1, wherein the members are manually engageable in an assembly such that removal of the first member from the second member requires a force of at least 5000 N.

7. A spinal implant system as recited in claim 1, wherein the inner surface of each of the first members includes a wall defining a groove, and further comprising a band that is expandable within the groove, the second member being manually engageable with the band to connect the members.

8. A spinal implant system as recited in claim 7, wherein the band is expandable between a capture orientation and an expanded orientation.

9. A spinal implant system as recited in claim 7, wherein the band is expandable with a force in a range of 2 through 50 N.

10. A spinal implant system as recited in claim 7, wherein the band is expandable with a force in a range of 5 through 10 N.

11. A spinal implant system as recited in claim 7, wherein the band is a circumferential ring that defines a gap.

12. A spinal implant system as recited in claim 11, wherein the gap defines a dimension that is less than at least one dimension of the ring.

13. A spinal implant system as recited in claim 11, wherein the gap defines a slot thickness that is less than a height and a width of the ring.

14. A spinal implant system as recited in claim 1, wherein the inner surface of each of the first members includes a wall defining a groove, the groove including a first circumferential channel and a second circumferential channel, and further comprising a band being disposed in the first circumferential channel in a capture orientation and in the second circumferential channel in an expanded orientation.

15. A spinal implant system as recited in claim 14, wherein the second member is axially translatable relative to the first member to expand the band.

16. A spinal implant system as recited in claim 1, further comprising a crown disposed with the implant cavity and engageable with the second member.

17. A method of assembly for a bone fastener, the method comprising the steps of:
selecting a first member from a plurality of alternate first members for connection with a second member to comprise a bone fastener having a selected movement, the second member including a mating element engageable with a first member such that the second member is interchangeable with the plurality of first members; and
manually engaging the second member with the selected first member to connect the members in a non-instrumented assembly.

18. A method as recited in claim 17, wherein the step of manually engaging includes snap fitting the first member with the second member.

19. A method as recited in claim 17, wherein the step of manually engaging includes connecting the first member with the second member in a pop-on assembly.

20. A spinal implant system comprising:
a plurality of alternate implant receivers; and
a bone screw shaft including a head engageable with an implant receiver such that the shaft is compatible with the plurality of implant receivers,
wherein an implant receiver is selected for connection with the shaft to comprise a bone fastener having a selected movement and the head is engageable with the implant receiver in a manual and non-instrumented assembly of the bone fastener.
